(19) United States
(12) Plant Patent Application Publication
(10) Pub. No.: US 2025/0261572 P1
(43) Pub. Date: Aug. 14, 2025

(54) Zoysia japonica PLANT NAMED '16-TZ-14114'

(50) ;

(71) Applicants: University of Georgia Research Foundation, Inc., Athens, GA (US); Patten Seed Company, DBA Super Sod, Fort Valley, GA (US)

(72) Inventor: Brian Schwartz, Tifton, GA (US)

(21) Appl. No.: 19/052,226

(22) Filed: Feb. 12, 2025

Related U.S. Application Data
(60) Provisional application No. 63/101,186, filed on Feb. 12, 2024.

(51) Int. Cl. A01H 6/46; A01H 5/00; A01H 5/12

(52) U.S. Cl. PLT/390

(57) ABSTRACT

A new and distinct plant of Zoysia plant named ‘16-TZ-14114’, characterized by its wide leaf texture and vigorous growth, which results in fast establishment after planting. The Zoysia plant named ‘16-TZ-14114’ is an asexually reproduced, tetraploid plant is reliably propagated vegetatively, although it is not seed or pollen sterile.

Genus and species: Latin name: Zoysia japonica.

Variety denomination: Variety denomination: ‘16-TZ-14114’.

### BACKGROUND OF THE INVENTION

The present invention relates to a new and distinct plant of zoysiagrass, botanically known as Zoysia japonica, and hereinafter referred to by the plant name ‘16-TZ-14114’.

The new Zoysia plant ‘16-TZ-14114’ was collected and evaluated as a product of a breeding program conducted by the inventor in Tifton, Georgia. The objective of the zoysiagrass breeding program is to create new plant plants with improved commercial qualities, e.g., that have wide leaf texture and vigorous growth, which results in fast establishment after planting. This plant is commercially important for its excellent turfgrass quality, increased seed head production, increased nematode resistance, and other qualities, which are enumerated herein.

### BRIEF SUMMARY OF THE INVENTION

The new Zoysia plant ‘16-TZ-14114’ has not been observed under all possible environmental conditions. The phenotype may vary somewhat with variations in environment and cultural practices such as temperature, water and fertility levels, soil types, and light intensity without, however, any variance in genotype. The following traits have been repeatedly observed and are determined to be the unique and distinguishing characteristics of the new Zoysia plant ‘16-TZ-14114’. In combination, these traits set ‘16-TZ-14114’ apart from all other existing varieties of Zoysia known to the inventor.

‘16-TZ-14114’ is a Zoysia japonica turfgrass that exhibits fast establishment (Table 1), vigorous growth and quick recovery, and wide leaf texture (Table 2). These characteristics make it an ideal turfgrass for lawns, commercial landscapes, and roadsides.

TABLE 1
Percent green plot coverage for select zoysiagrass
plants during field establishment as measured
each month during 2021 in in Tifton, GA.

| | Mar | | Apr | | May | | June | | July | |
|---|---|---|---|---|---|---|---|---|---|---|
| ‘16-TZ-14114’ | 1.5 | a | 6.6 | a | 26.3 | a | 32.7 | a | 61.2 | a |
| ‘Diamond’ (US PP10636P) | 0.4 | b | 2.2 | b | 8.8 | b | 24.1 | ab | 58.6 | a |
| ‘Palisades’ (US PP11515) | 0.2 | b | 0.9 | b | 7.6 | b | 12.9 | b | 42.1 | a |
| ‘Zeon’ (US PP13166) | 0.1 | b | 0.3 | b | 4.8 | b | 10.9 | b | 38.7 | a |

Means followed within columns by the same letter are not significantly different at K = 100 (approximately P = 0.05) according to the Waller-Duncan LSD.

TABLE 2
Leaf measurements of selected zoysiagrass
plants measured under greenhouse
conditions during 2020 in Tifton, GA.

| | Leaf | |
|---|---|---|
| Genotype | width (mm) | |
| ‘SS-500’ Empire ™ (US PP11466) | 4.12 | a |
| ‘16-TZ-14114’ | 4.11 | a |
| ‘Meyer’ (not patented) | 3.38 | b |
| ‘Zeon’ (US PP13166) | 2.07 | c |
| ‘BA-305’ Emerald ™ (US PP18415) | 1.89 | cd |
| ‘Diamond’ (US PP10636P) | 1.68 | d |

Means followed within columns by the same letter are not significantly different at K = 100 (approximately P = 0.05) according to the Waller-Duncan LSD.

Morphological comparisons of ‘16-TZ-14114’ with ‘Diamond’, ‘Emerald’, ‘Empire’, ‘Meyer’, and ‘Zeon’ in greenhouse trials show that ‘16-TZ-14114’ is most like ‘Empire’, but significantly different than ‘Empire’ in leaf color (Table 2), internode length (Table 3), and raceme size (Table 4). ‘16-TZ-14114’ is significantly different from ‘Meyer’, ‘Zeon’, ‘Emerald’, and ‘Diamond’ in leaf width and length (Table 2), as well as raceme and peduncle size, and number of spikelets per raceme (Table 4). Fewer differences were observed for stolon characteristics.

### BRIEF DESCRIPTION OF THE DRAWINGS

The accompanying colored photographic illustrations show the overall appearance and distinct characteristics of the new cultivar of Zoysia japonica ‘16-TZ-14114” showing the colors as true as possible. Colors in the photographs may differ slightly from the color values cited in the detailed botanical description, which accurately describes the colors of the new Genus species ‘cultivar name’.

FIG. 1 is a view of 16-TZ-14114.

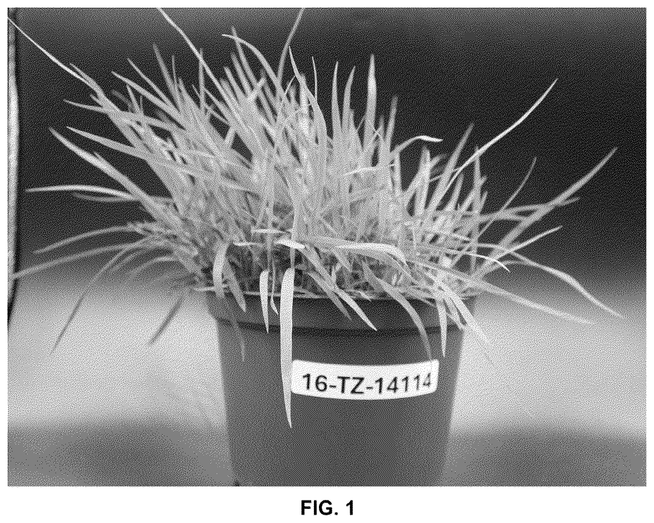

FIG. 2 is a second view of 16-TZ-14114.

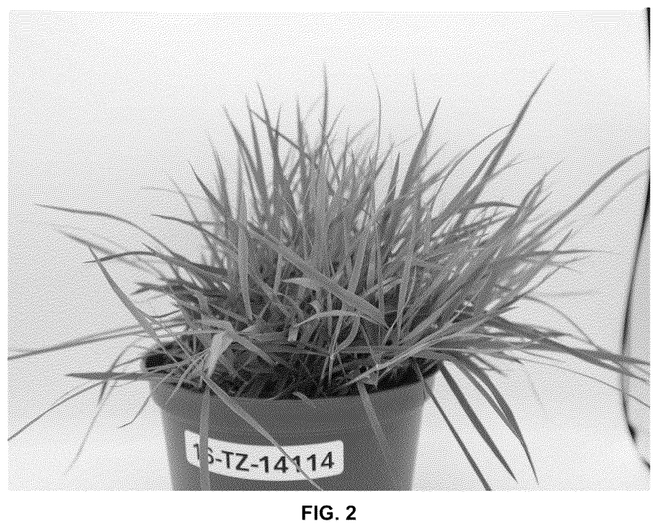

FIG. 3 is a third view of 16-TZ-14114.

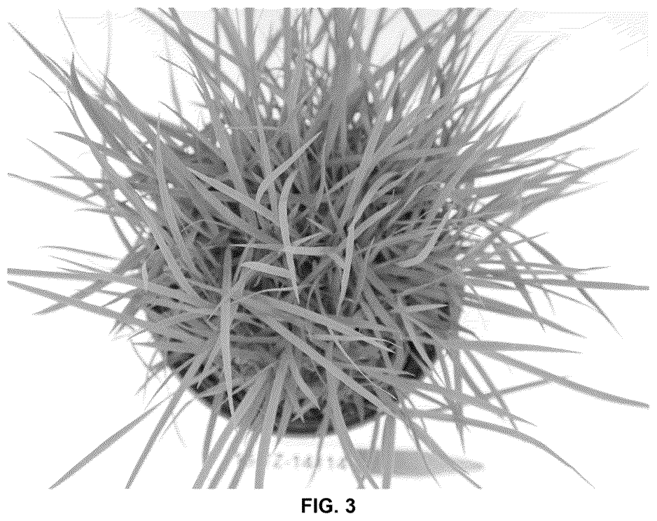

### DETAILED BOTANICAL DESCRIPTION

The following traits have been consistently observed in the original plant of this new variety and in asexually propagated progeny grown in Tifton, Georgia, and, to the best knowledge of the inventors, their combination forms the unique characteristics of the new variety ‘16-TZ-14114’. Sod, sprigs, or plugs was the type of asexual reproduction used.

The following parents were used to produce the plant:
a) 2016: Selected from within an uncultivated population of mixed grasses;
b) 2016-2022: sod farm research trials for sod establishment, tensile strength, and sod re-growth (Super-Sod, Lakeland & Super-Sod, Marshallville);
c) 2017-Present: University research trials for general turfgrass performance (UGA);
d) 2018-Present: University research trials for winter-kill (NC State, Purdue, Texas Tech, and UGA);
e) 2018-2020: University and sod farm research trials of golf rough performance (NC State, Purdue, UGA, and Evergreen Turf, Arizona & American Sod Farms, California);
f) 2018-2022: National Turfgrass Evaluation Program-2018 National Low Input Warm-Season Trial (Miss State, NC State, NM State, OK State, TAMU, UF, and Utah State);
g) 2019-Present: University research trials for drought tolerance (NC State, OK State, TAMU, UC-Riverside, UF, and UGA);
h) 2019-Present: Independent research trials for shade tolerance (Good Hope, GA);

The following observations, measurements, and values describe the new Zoysia plant ‘16-TZ-14114’ grown in Tifton, Georgia. The following traits have been consistently observed and, to the best knowledge of the inventor, their combination forms the unique characteristics of the new variety ‘16-TZ-14114’. During the growing of the plants, typical day temperatures in Tifton, GA ranged from 69° F. to 99° F., and typical night temperatures ranged from 47° F. to 70° F.

Throughout this specification, color names beginning with a small letter signify that the name of that color, as used in common speech, is aptly descriptive. Color names beginning with a capital letter designate values based upon The R.H.S. Colour Chart, 5^{th} edition published by the Royal Horticultural Society, London, England in 2007, except where general terms of ordinary dictionary significance are used.

The following observations, measurements, and values describe plants of the Zoysia plant ‘16-TZ-14114’.

‘16-TZ-14114’ has demonstrated quick establishment, vigorous growth, and quick recovery from injury. In field trials planted at The University of Georgia Tifton Campus, plots of ‘Zeon’, ‘Diamond’, ‘Palisades’, and ‘16-TZ-14114’ were evaluated for the percentage of ground covered by vegetation during each month in 2021 (Table 1). ‘16-TZ-14114’ established rapidly and by May plots it averaged 26.3% coverage against the next highest ‘Diamond’ at 8.8%. In the following months during 2021, ‘16-TZ-14114’ continued to out-grow the other plants, but the spread was not statistically faster.

The leaf of ‘16-TZ-14114’ is wide (4.11 mm), has rolled vernation, and is 77.59 mm long (Table 2). The adaxial (upper) and abaxial (lower) leaf surfaces are smooth with a few trichomes dispersed along the leaf margins. The leaf width (texture) of ‘16-TZ-14114’ is similar to ‘Empire’, while significantly wider than ‘Meyer’, ‘Zeon’, ‘Emerald’, and ‘Diamond’. The leaf color of ‘16-TZ-14114’ most fits the Green Group 137C of The Royal Horticulture Society (R.H.S.) Colour Chart, 5^{th} Edition and was also determined to have a dark green color index of 0.66 through the analysis of digital images taken under controlled lighting. This color was similar to that of ‘Meyer’, ‘Zeon’, ‘Emerald’, and ‘Diamond’, but lighter than the leaf color of ‘Empire’.

The leaf sheath at the collar region of ‘16-TZ-14114’ measured 3.56 mm (σ=0.19) and had the R.H.S. color 145B. The ligule was hairy with a 157D R.H.S. color and auricles were not pronounced. Overall stolon length of ‘16-TZ-14114’ averaged 24.38 mm (Table 3) and had the R.H.S. color 150D. Stolon internode length for ‘16-TZ-14114’ was 17.28 mm and was significantly shorter than found in ‘Empire’, while longer than those of ‘Zeon’, ‘Emerald’, or ‘Diamond’.

‘16-TZ-14114’ has a raceme inflorescence that averaged 28.19 mm in length (Table 4) and was significantly shorter than observed on ‘Empire’, while being longer than those of ‘Meyer’, ‘Zeon’, ‘Emerald’, or ‘Diamond’. ‘16-T-14114’ peduncle length was 62.39 mm and best fit R.H.S. color 143B. Its spikelets measured 3.35 mm long (σ=0.17) and had the R.H.S. color 145D. Glumes were 0.21 mm (σ=0.02) in length with a 145D R.H.S. color, while the lemma best fit a R.H.S. color of 145C and was 0.29 mm long (σ=0.02).

TABLE 3
Stolen measurements of selected zoysiagrass plants measured
under greenhouse conditions during 2020 in Tifton, GA.

| | | | | | Stolon | |
|---|---|---|---|---|---|---|
| | | | | | internode | |
| | Stolon | | Internode | | diameter | |
| Genotype | length (cm) | | length (mm) | | (mm) | |
| ‘SS-500’ Empire ™ (US PP11466) | 27.05 | a | 25.52 | a | 2.47 | a |
| ‘16-TZ-14114’ | 24.38 | ab | 17.28 | b | 2.07 | ab |
| ‘Meyer’ (not patented) | 23.49 | ab | 16.68 | b | 1.90 | ab |
| ‘Zeon’ (US PP13166) | 23.31 | ab | 12.24 | c | 1.84 | ab |
| ‘BA-305’ Emerald ™ | 18.68 | bc | 11.33 | c | 1.50 | ab |
| (US PP18415) | | | | | | |
| ‘Diamond’ (US PP10636P) | 14.71 | c | 9.62 | c | 1.16 | b |

Means followed within columns by the same letter are not significantly different at K = 100 (approximately P = 0.05) according to the Waller-Duncan LSD.

TABLE 4
Inflorescence characteristics of selected
zoysiagrass plants measured under greenhouse
conditions during 2020 in Tifton, GA.

| | | | | | Spikelets | |
|---|---|---|---|---|---|---|
| | | | | | per | |
| | Raceme | | Peduncle | | inflorescence | |
| Genotype | size (mm) | | length (mm) | | (number) | |
| ‘SS-500’ Empire ™ | 38.67 | a | 65.30 | a | 33.2 | a |
| (US PP11466) | | | | | | |
| ‘16-TZ-14114’ | 28.19 | b | 62.39 | a | 32.0 | a |
| ‘Meyer’ (not patented) | 23.71 | c | 45.50 | b | 27.5 | b |
| ‘Zeon’ (US PP13166) | 16.88 | d | 35.62 | c | 15.6 | c |
| ‘BA-305’ Emerald ™ | 12.41 | e | 18.09 | d | 15.3 | c |
| (US PP18415) | | | | | | |
| ‘Diamond’ | 11.23 | d | 13.43 | d | 6.4 | d |
| (US PP10636P) | | | | | | |

Means followed within columns by the same letter are not significantly different at K = 100 (approximately P = 0.05) according to the Waller-Duncan LSD.

TABLE 5
16-TZ-141-14 Measurements

| Task | Type | Description | AVG | SD |
|---|---|---|---|---|
| D | Color | Inflorescence peduncle | 143b | . |
| D | Color | Inflorescence floret | 145d | . |
| D | Color | Inflorescence palea | 145c | . |
| D | Color | Inflorescence lemma (fertile) | 145d | . |
| D | Color | Inflorescence lemma (sterile) | n/a | . |
| D | Color | Inflorescence glume (lower) | n/a | . |
| D | Color | Inflorescence glume (upper) | n/a | . |
| D | Color | Inflorescence awn | n/a | . |
| E | Size | Inflorescence floret | 3.345 | 0.166 |
| E | Size | Inflorescence palea | 0.288 | 0.024 |
| E | Size | Inflorescence lemma (fertile) | 0.214 | 0.019 |
| E | Size | Inflorescence lemma (sterile) | n/a | . |
| E | Size | Inflorescence glume (lower) | n/a | . |
| E | Size | Inflorescence glume (upper) | n/a | . |
| E | Size | Inflorescence awn | n/a | . |
| F | Color | Stolon | 150d | . |
| F | Color | Ligule | 157d | . |
| F | Color | Sheath | 145b | . |
| F | Color | Collar | 149d | . |
| G | Size | Sheath | 3.557 | 0.192 |
| H | Type | Ligule | hairy | . |
| | Type | Auricle | absent | . |
| J | Type | Collar (continuous, | 0.141 | 0.033 |
| | | constricted at midrib) | | . |
| K | Description | Upper (adaxial) leaf | smooth | . |
| | | surface texture | | . |
| K | Description | Lower (abaxial) leaf | smooth | . |
| | | surface texture | | . |
| K | Description | Hairs on leaf edges | few | . |
| L | Designation | Foliar disease | No test, assumed | . |
| | | | Bipolaris | . |
| L | Designation | Root disease | Gaeumannomyces | . |
| | | | graminis | . |
| M | color | Anther | 145a | . |

What is claimed is:

1. A new and distinct plant of the Zoysia plant named Zoysia ‘16-TZ-14114’ as illustrated and described herein.
